SEQUENCE LISTING (12 sequences)

SEQ ID NO: 1 - DNA, 116 bp, synthetic construct
  source 1..116: mol_type = other DNA; note = PpepN promoter sequence
ctgtcagtagacagtttttttaataagttaaagaaaagatgtaatttttctttgtactcgaaattttctattcaatttgatataattatattaatactgaatatttaggagaaggc

SEQ ID NO: 2 - protein, 38 aa, synthetic construct
  source 1..38: note = Usp45 secretion signal peptide sequence
MKKKIISAILMSTVILSAAAPLSGVYADTNSDIAKQDA

SEQ ID NO: 3 - protein, 114 aa, African swine fever virus
  source 1..114: note = major capsid protein p72 of African swine fever virus, partial sequence
DYKDDDDKACHSSWQDAPIQGTSQMGAHGQLQTFPRNGYDWDNQTPLEGAVYTLVDPFGRPIVPGTKNAYRNLVYYCEYPGERLYENVRFDVNGNSLDEYSSDVTTLVHHHHHH

SEQ ID NO: 4 - DNA, 114 bp, synthetic construct
  source 1..114: mol_type = other DNA; note = Coding sequence of Usp45 secretion signal peptide
atgaaaaaaaagattatctcagctattttaatgtctacagtgatactttctgctgcagccccgttgtcaggtgtttacgctgacacaaactcagatattgctaaacaagatgcg

SEQ ID NO: 5 - DNA, 345 bp, synthetic construct
  source 1..345: mol_type = other DNA; note = Coding sequence of P72 protein, codon optimized
gattacaaggatgacgacgataaggcctgccactcgagttggcaagatgcaccaattcaaggcacgagtcagatgggcgcacatggtcagttacagacttttccacgcaatgggtatgactgggataatcagacaccgcttgaaggcgctgtttatacgttagttgatccatttggccgcccaatcgtgccaggcaccaagaatgcttatcgcaacttagtgtattattgtgagtatcccggagagcggctatatgaaaacgtccgttttgatgttaacggcaattcacttgacgagtactcctctgatgtcactactttggtacaccaccaccaccaccactaa

SEQ ID NO: 6 - DNA, 41 bp, synthetic construct
  source 1..41: mol_type = other DNA; note = forward primer sequence for PpepN promoter
ataaggaggcactcaccatggctgtcagtagacagtttttt

SEQ ID NO: 7 - DNA, 52 bp, synthetic construct
  source 1..52: mol_type = other DNA; note = reverse primer sequence for PpepN promoter
gagataatcttttttttcatgccttctcctaaatattcagtattaatataat

SEQ ID NO: 8 - DNA, 40 bp, synthetic construct
  source 1..40: mol_type = other DNA; note = forward primer sequence for Usp45 signal peptide gene
atattcggaggaattttgaaatgaaaaaaaagattatctc

SEQ ID NO: 9 - DNA, 40 bp, synthetic construct
  source 1..40: mol_type = other DNA; note = reverse primer sequence for Usp45 signal peptide gene
tcgtcgtcatccttgtaatccgcatcttgtttagcaatat

SEQ ID NO: 10 - DNA, 24 bp, synthetic construct
  source 1..24: mol_type = other DNA; note = forward primer sequence for P72 gene
gattacaaggatgacgacgataag

SEQ ID NO: 11 - DNA, 21 bp, synthetic construct
  source 1..21: mol_type = other DNA; note = reverse primer sequence for P72 gene
ttagtggtggtggtggtggtg

SEQ ID NO: 12 - DNA, 575 bp, synthetic construct
  source 1..575: mol_type = other DNA; note = a spliced sequence of PpepN promoter sequence, coding sequence of the Usp45 secretion signal peptide and coding sequence of P72 protein
ctgtcagtagacagtttttttaataagttaaagaaaagatgtaatttttctttgtactcgaaattttctattcaatttgatataattatattaatactgaatatttaggagaaggcatgaaaaaaaagattatctcagctattttaatgtctacagtgatactttctgctgcagccccgttgtcaggtgtttacgctgacacaaactcagatattgctaaacaagatgcggattacaaggatgacgacgataaggcctgccactcgagttggcaagatgcaccaattcaaggcacgagtcagatgggcgcacatggtcagttacagacttttccacgcaatgggtatgactgggataatcagacaccgcttgaaggcgctgtttatacgttagttgatccatttggccgcccaatcgtgccaggcaccaagaatgcttatcgcaacttagtgtattattgtgagtatcccggagagcggctatatgaaaacgtccgttttgatgttaacggcaattcacttgacgagtactcctctgatgtcactactttggtacaccaccaccaccaccactaa

(19) United States
(12) Patent Application Publication
(10) Pub. No.: US 2025/0375513 A1
(43) Pub. Date: Dec. 11, 2025

(54) DUAL-PROMOTER RECOMBINANT VECTOR EXPRESSING IMMUNOGENIC PROTEIN OF AFRICAN SWINE FEVER VIRUS (ASFV), RECOMBINANT BACTERIUM, AND USE THEREOF

(71) Applicant: CHANGSHA LVYE BIOTECHNOLOGY CO., LTD., Changsha (CN)

(72) Inventors: Jiayi HE, Changsha (CN); Wei DONG, Changsha (CN); Fan LIAO, Changsha (CN); Qing LI, Changsha (CN); Yuanyuan ZHU, Changsha (CN); Xiaohong XIE, Changsha (CN); Lixin WEN, Changsha (CN)

(73) Assignee: CHANGSHA LVYE BIOTECHNOLOGY CO., LTD., Changsha (CN)

(21) Appl. No.: 19/070,579

(22) Filed: Mar. 5, 2025

(30) Foreign Application Priority Data
Jun. 5, 2024 (CN) 202410719629.5

(51) Int. Cl. A61K 39/12; A61K 39/00; A61P 31/20; C12N 1/20; C12N 7/00; C12N 15/74; C12R 1/46

(52) U.S. Cl. CPC A61K 39/12; A61P 31/20; C12N 1/20; C12N 7/00; C12N 15/746; A61K 2039/523; A61K 2039/53; A61K 2039/545; C12N 2710/12034; C12R 2001/46

(57) ABSTRACT

A dual-promoter recombinant vector expressing an immunogenic protein of an African swine fever virus (ASFV), a recombinant bacterium, and use thereof are provided. A recombinant gene consisting of a PpepN promoter, a coding sequence of a Usp45 secretion signal peptide, and a coding sequence of the immunogenic protein of the ASFV that are ligated in sequence is inserted downstream of a promoter in an original vector to obtain the dual-promoter recombinant vector expressing the immunogenic protein of the ASFV.

### CROSS REFERENCE TO THE RELATED APPLICATIONS

This application is based upon and claims priority to Chinese Patent Application No. 202410719629.5, filed on Jun. 5, 2024, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named WGJB0208_Sequence_Listing.xml, created on Feb. 21, 2025, and is 15,422 bytes in size.

### TECHNICAL FIELD

The present disclosure belongs to the technical field of gene recombination, and specifically relates to a dual-promoter recombinant vector expressing an immunogenic protein of an African swine fever virus (ASFV), a recombinant bacterium, and use thereof.

### BACKGROUND

African swine fever (ASF) is an acute, highly contagious, and highly lethal infectious disease characterized by pathological symptoms such as persistent high fever, systemic bleeding, enlarged spleen, and dysfunction in respiratory and nervous systems caused by the African swine fever virus (ASFV) that resides in the blood, tissue fluid, and internal organs of infected pigs. The ASFV is a DNA virus with a particle diameter of 175 nm to 215 nm, are icosahedral in symmetry, and have an envelope.

ASF is a highly contagious disease. After entering the pig's body through the mouth and upper respiratory system, where infection occurs in the nasopharynx or tonsils, ASFV rapidly spreads to the mandibular lymph nodes and invades the whole body through lymph fluid and blood. ASFV only infects pigs and does not infect other animals, indicating that there are specific receptors in cells of the pig body to this virus. Blocking the virus from invading the body through the surface of the mucosal tissue is a key point in the effective control of viral epidemic diseases. Although many attempts have been made to study inactivated vaccines, live attenuated vaccines, and subunit vaccines for the ASF, the results have not been satisfactory. There is currently no commercialized vaccine against ASFV. At present, the safest, most economical and most effective means of ASFV control is biosafety control, and a principle of the biosafety control is to prevent the virus from coming into contact with the body. However, none of the existing methods can guarantee that the virus and the body no longer come into contact.

The present disclosure has the same purpose as the previously filed patent application CN111454982A, and both of which is to prepare an oral preparation that can be used to prevent ASFV. However, the recombinant bacterium constructed in CN111454982A has low expression level of target protein and poor stability.

### SUMMARY

A purpose of the present disclosure is to provide a dual-promoter recombinant vector expressing an immunogenic protein of an ASFV, a recombinant bacterium, and use thereof. In the present disclosure, a target protein may be expressed by using a dual-promoter combination without adding Nisin in a desirable stability, and adding the Nisin, an expression level of the target protein is greatly improved.

The present disclosure provides a dual-promoter recombinant vector expressing an immunogenic protein of an ASFV, including an original vector and a recombinant gene; where the original vector includes a pNZ8149 vector; the recombinant gene includes a PpepN promoter, a coding sequence of a Usp45 secretion signal peptide, and a coding sequence of the immunogenic protein of the ASFV that are ligated in sequence; and the PpepN promoter includes a nucleotide sequence set forth in SEQ ID NO: 1 or a complementary sequence thereof; and
the recombinant gene is located downstream of a promoter in the original vector.

In some embodiments, the Usp45 secretion signal peptide has an amino acid sequence set forth in SEQ ID NO: 2; and

the immunogenic protein of the ASFV includes an African swine fever (ASF) p72 protein.

In some embodiments, the ASF p72 protein has an amino acid sequence set forth in SEQ ID NO: 3.

In some embodiments, the recombinant gene is located between restriction sites Nco I and Xbal I of the original vector.

The present disclosure further provides a recombinant bacterium, including the dual-promoter recombinant vector; where a basic bacterium of the recombinant bacterium includes one or more of a normal intestinal flora.

In some embodiments, the normal intestinal flora is selected from the group consisting of Bifidobacterium, Lactococcus, Lactobacillus, and Streptococcus.

In some embodiments, the Lactococcus includes Lactococcus lactis NZ3900.

The present disclosure further provides a microbial preparation for preventing ASFV infection, including the recombinant bacterium described above as an active ingredient.

In some embodiments, the recombinant bacterium has a viable count of 2×10¹⁰ CFU/mL to 3×10¹⁰ CFU/mL.

The present disclosure further provides use of the dual-promoter recombinant vector, the recombinant bacterium, or the microbial preparation described above in preparation of a drug for preventing ASFV infection.

The present disclosure provides a dual-promoter recombinant vector expressing an immunogenic protein of an ASFV, including an original vector and a recombinant gene; where the original vector includes a pNZ8149 vector; the recombinant gene includes a PpepN promoter, a coding sequence of a Usp45 secretion signal peptide, and a coding sequence of the immunogenic protein of the ASFV that are ligated in sequence; and the recombinant gene is located downstream of a promoter in the original vector. The present disclosure constructs a recombinant expression vector of the immunogenic protein of the ASFV. The exocrine expression of the immunogenic protein of the ASFV may be realized by using the Usp45 secretion signal peptide in the recombinant expression vector; a dual-promoter combination of the PpepN promoter and the promoter in the original vector (P NisinA) greatly improves an expression level of a target protein, and the target protein can still be detected by WB experiment even in the absence of a Nisin inducer. The recombinant bacterium containing the dual-promoter recombinant vector has been proved to have strong stability, and the target protein may be detected even after passage to the 6th generation by the results of subculture experiment.

Further, the recombinant bacterium containing the dual-promoter recombinant vector of the present disclosure may be used to prepare a microbial preparation for preventing ASFV infection. In the present disclosure, after the pig takes the microbial preparation, an antigen protein (p72 protein) secreted by Lactococcus lactis adheres to surface of the cell membrane at the mucosa site of the body and forms an antigen protein membrane on the surface of the mucosa. The antigen protein binds to a virus binding site on the target cell, blocks a receptor site to virus protein on the surface of the cell membrane at the mucosa site, and plays a role of ecological occupation. When the live virus in the environment invades the body, the virus binding site on the target cell has been blocked by the antigen protein biofilm. This process prevents the virus from binding to the virus binding site on the target cell, thereby effectively blocking the virus from binding to the receptor on the cell surface, and preventing ASF. Compared with vaccines, the oral preparation in the present disclosure is safer, more effective, and faster.

### BRIEF DESCRIPTION OF THE DRAWINGS

To describe the technical solutions in examples of the present disclosure or in the prior art more clearly, the accompanying drawings required in the examples are briefly described below. Apparently, the accompanying drawings in the following description show merely some examples of the present disclosure, and other drawings can still be derived from these accompanying drawings by those of ordinary skill in the art without creative efforts.

FIGS. 1A-1C are images showing amplification of a target fragment detected by gel electrophoresis;

FIG. 2 shows a map of the pNpepN-ASFV-P72 plasmid;

FIG. 3 is an image showing PCR identification results of whether a strain is a positive strain detected by gel electrophoresis;

FIG. 4 is an image showing expression of the P72 target protein by Western Blot; and

FIG. 5 is an image showing a stability of the P72 target protein detected by Western Blot.

### DETAILED DESCRIPTION OF THE EMBODIMENTS

The present disclosure provides a dual-promoter recombinant vector expressing an immunogenic protein of an ASFV, including an original vector and a recombinant gene; where the original vector includes a pNZ8149 vector; the recombinant gene includes a PpepN promoter, a coding sequence of a Usp45 secretion signal peptide, and a coding sequence of the immunogenic protein of the ASFV that are ligated in sequence; and the PpepN promoter includes a nucleotide sequence set forth in SEQ ID NO: 1 and a complementary sequence to the nucleotide sequence set forth in SEQ ID NO: 1; and the recombinant gene is located downstream of a promoter in the original vector.

In the present disclosure, the PpepN promoter in the dual-promoter recombinant vector can initiate the expression of a downstream gene and can be used for the efficient expression of endogenous or exogenous proteins in prokaryotes. There is no particular limitation on a source of the PpepN promoter, and a promoter sequence synthesis method known in the art can be used.

In the present disclosure, the recombinant gene is preferably located downstream of a P NisinA promoter in the original vector, and more preferably the recombinant gene is located between restriction sites Nco I and Xbal I of the original vector. A dual-promoter combination consisting of the P NisinA promoter and the PpepN promoter in the original vector can still express a target protein without adding a Nisin inducer. The results of the subculture experiment prove that the recombinant bacterium containing the dual-promoter recombinant vector has strong stability, and the target protein can still be detected after subculture to the 6th generation. Moreover, after adding the Nisin inducer, the expression level of the target protein of the dual-promoter combination is greatly improved compared with that when the Nisin inducer is not added.

In the present disclosure, the Usp45 secretion signal peptide has an amino acid sequence preferably set forth in SEQ ID NO: 2, specifically: MKKKIISAILMSTVILSAAAPLSGVYADTNSDIAKQDA; the coding sequence of the Usp45 secretion signal peptide is preferably set forth in SEQ ID NO: 4. The Usp45 secretion signal peptide in the recombinant expression vector can realize exocrine expression of the immunogenic protein of the ASFV.

In the present disclosure, the immunogenic protein of the ASFV preferably includes an ASF p72 protein; the ASF p72 protein has an amino acid sequence preferably set forth in SEQ ID NO 3, specifically:

DYKDDDDKACHSSWQDAPIQGTSQMGAHGQLQTFPRNGYDWDNQTPLE
GAVYTLVDPFGRPIVPGTKNAYRNLVYYCEYPGERLYENVRFDVNGNS
LDEYSSDVTTLVHHHHHH;

the nucleotide sequence encoding the ASFV p72 protein is preferably set forth in SEQ ID NO: 5. The P72 protein is a major capsid protein of ASFV particles and plays a critical role in virus recognition, binding, and infection of host cells.

The present disclosure further provides a recombinant bacterium, including the dual-promoter recombinant vector; where a basic bacterium of the recombinant bacterium includes one or more of a normal intestinal flora.

In the present disclosure, the normal intestinal flora is preferably selected from the group consisting of Bifidobacterium, Lactococcus, Lactobacillus, and Streptococcus. Preferably, the Lactococcus includes Lactococcus lactis NZ3900. The Lactococcus lactis NZ3900 is food-grade. The food-grade Lactococcus lactis NZ3900 and its matching plasmid pNZ8149 are the only pair of matching operating systems in the current lactic acid bacteria expressing exogenous proteins that can use lactose as the sole carbon source to screen positive clones and do not contain any resistance genes, showing high safety. Compared with Lactobacillus casei in the prior patent CN111454982A, the Lactococcus lactis NZ3900 has a significantly shortened growth cycle, thereby reducing the culture time and cost.

The present disclosure further provides a microbial preparation for preventing ASFV infection, including the recombinant bacterium as an active ingredient. In the present disclosure, the microbial preparation is preferably an oral preparation; the recombinant bacterium preferably has a viable count of 2×10¹⁰ CFU/mL to 3×10¹⁰ CFU/mL, more preferably 3×10¹⁰ CFU/mL. The active ingredient of the microbial preparation can secrete and express viral immunogenic proteins, and there is no viral gene present, and no viral mutation occurs, showing desirable safety. The protective antigens secreted by the active ingredient of the microbial preparation act on the mucosal parts of the body surface and cover the surface of the mucous membrane where the target cells of ASFV are located; when the virus invades, the sites on the cells on the surface of the mucous membrane where it binds are occupied and blocked, thereby blocking the viral infection path, showing desirable effectiveness. The secretory protein expressed by Lactococcus lactis directly seizes the receptors that bind the virus on the target cells to block viral infection, without the need for an immune response process, which is rapid in response.

The present disclosure further provides use of the dual-promoter recombinant vector described in the above solutions, the recombinant bacterium, or the microbial preparation described in the above solutions in preparation of a drug for preventing ASFV infection.

In order to further illustrate the present disclosure, the dual-promoter recombinant vector and the recombinant bacterium expressing an immunogenic protein of an ASFV, and the use thereof provided by the present disclosure are described in detail below with reference to the accompanying drawings and examples, but the accompanying drawings and the examples should not be construed as limiting the protection scope of the present disclosure.

The reagents and test materials used in the examples of the present disclosure are all commercially available products. For example, restriction endonucleases Nco I and Xbal I are purchased from NEB company; DNA Marker DL5000 and DNA Marker DL2000 are purchased from Beijing Solarbio Science & Technology Co., Ltd.; Taq enzyme, Agarose Gel DNA Purification Kit, and Mini BEST Plasmid Purification Kit are purchased from Nanjing Vazyme Biotech Co., Ltd. Nisin, GM17 medium, and MRS medium are all purchased from Beijing Coolaber Biotechnology Co. The cloning vector pNZ8149, Lactococcus lactis NZ9000 and Lactococcus lactis NZ3900 are provided by Bio Sci Bio. The pVE5523 vector is synthesized by Nanjing GenScript Biotech Co., Ltd.

### Example 1

### Acquisition of the ASFV P72 Gene Sequence and the PpepN-Usp45-P72 Target Fragment

The P72 protein is a major capsid protein of an ASFV particle and plays a critical role in various processes such as recognition, binding, and infection of a virus to a host cell. According to the amino acid sequence of the ASFV P72 protein (GenBank: QID90230), nucleic acid sequence for part of the conserved region was selected and sent to Sangon Biotech (Shanghai) Co., Ltd. for codon optimization synthesis to obtain a P72 gene fragment having a base sequence set forth in SEQ ID NO: 5.

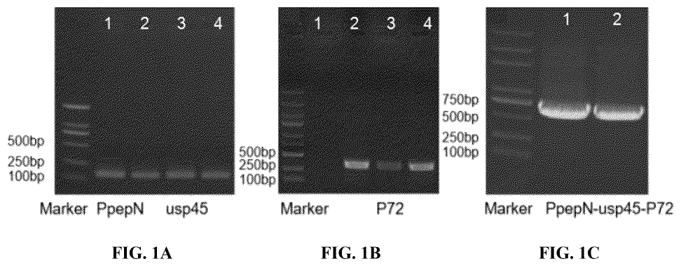

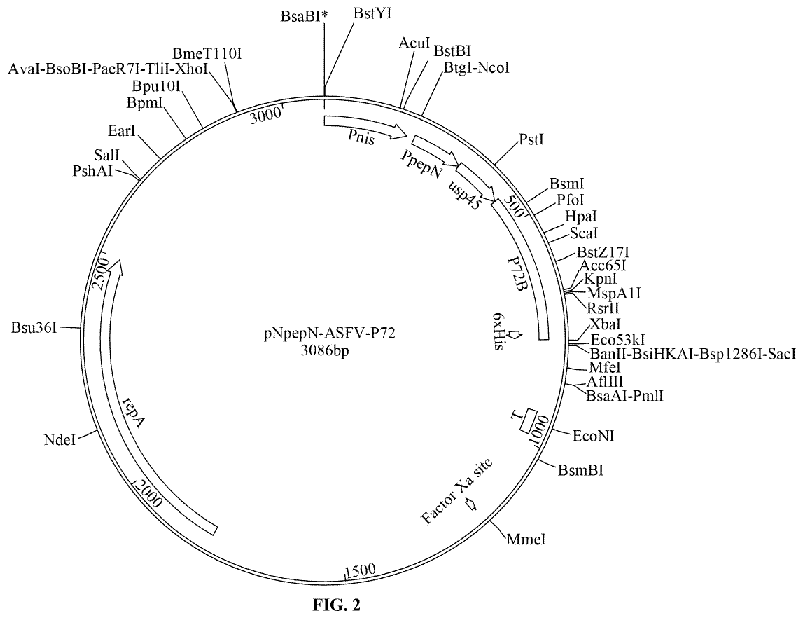

Primers were designed for amplification of the PpepN promoter fragment using Lactococcus lactis NZ9000 as a template, of the coding sequence of Usp45 secretion signal peptide using the pVE5523 vector as a template, and of the coding sequence of P72 protein using the synthetic P72 gene as a template. The electrophoresis results of the amplified products were shown in FIG. 1A and FIG. 1B, where lanes 1 and 2 represent the PpepN promoter, and lanes 3 and 4 represent the coding sequence of Usp45 secretion signal peptide in FIG. 1A; lanes 2, 3, and 4 in FIG. 1B represent the coding sequence of P72 protein. The target fragment was purified using an Omega gel extraction kit.

The amplified nucleotide sequence of the PpepN promoter fragment was set forth in SEQ ID NO: 1; the forward and reverse primers for amplifying the coding sequence of the PpepN promoter fragment were PpepN-F and PpepN-R, respectively. The amplified coding sequence of the Usp45 secretory protein was set forth in SEQ ID NO: 4; the forward and reverse primers for amplifying the coding sequence of the Usp45 secretion signal peptide were Usp45-F and Usp45-R, respectively. The amplified coding sequence of the P72 protein was set forth in SEQ ID NO: 5; the forward and reverse primers for amplifying the coding sequence of the P72 protein were P72-F and P72-R, respectively. Specific sequences were as follows:

PpepN promoter sequence:
(SEQ ID NO: 1)
5′-CTGTCAGTAGACAGTTTTTTTAATAAGTTAAAGAAAAGATGTAATTTTTCTT
TGTACTCGAAATTTTCTATTCAATTTGATATAATTATATTAATACTGAATATTTAGGAGAA
GGC-3′;
PpepN-F:
(SEQ ID NO: 6)
5′-ATAAGGAGGCACTCACCATGGCTGTCAGTAGACAGTTTTTT-3′;
PpepN-R:
(SEQ ID NO: 7)
5′-GAGATAATCTTTTTTTTCATGCCTTCTCCTAAATATTCAGTATTAATATAAT-3′;
Coding sequence of Usp45 secretion signal peptide:
(SEQ ID NO: 4)
5′-ATGAAAAAAAAGATTATCTCAGCTATTTTAATGTCTACAGTGATACTTTCTGC
TGCAGCCCCGTTGTCAGGTGTTTACGCTGACACAAACTCAGATATTGCTAAACAAGAT
GCG-3′;
Usp45F-:
(SEQ ID NO: 8)
5′-ATATTCGGAGGAATTTTGAAATGAAAAAAAAGATTATCTC-3′;
Usp45-R:
(SEQ ID NO: 9)
5′-TCGTCGTCATCCTTGTAATCCGCATCTTGTTTAGCAATAT-3′;
Coding sequence of P72 protein:
(SEQ ID NO: 5)
5′-GATTACAAGGATGACGACGATAAGGCCTGCCACTCGAGTTGGCAAGATGCA
CCAATTCAAGGCACGAGTCAGATGGGCGCACATGGTCAGTTACAGACTTTTCCACGC
AATGGGTATGACTGGGATAATCAGACACCGCTTGAAGGCGCTGTTTATACGTTAGTTG
ATCCATTTGGCCGCCCAATCGTGCCAGGCACCAAGAATGCTTATCGCAACTTAGTGTA
TTATTGTGAGTATCCCGGAGAGCGGCTATATGAAAACGTCCGTTTTGATGTTAACGGC
AATTCACTTGACGAGTACTCCTCTGATGTCACTACTTTGGTACACCACCACCACCACC
ACTAA-3′;
P72-F:
(SEQ ID NO: 10)
5′-GATTACAAGGATGACGACGATAAG-3′;
P72-R:
(SEQ ID NO: 11)
5′-TTAGTGGTGGTGGTGGTGGTG-3′.

The PpepN, Usp45, and P72 fragments were spliced into a long fragment PpepN-Usp45-P72 by overlapping PCR. A sequence of the spliced PpepN-Usp45-P72 long fragment was set forth in SEQ ID NO: 12, where 0 bp to 116 bp was the PpepN promoter sequence, 117 bp to 230 bp was the coding sequence of the Usp45 secretion signal peptide, and 231 bp to 575 bp was the coding sequence of P72 protein.

Coding sequence of PpepN-Usp45-P72:
(SEQ ID NO: 12)
5′-CTGTCAGTAGACAGTTTTTTTAATAAGTTAAAGAAAAGATGTAATT
TTTCTTTGTACTCGAAATTTTCTATTCAATTTGATATAATTATATTAAT
ACTGAATATTTAGGAGAAGGCATGAAAAAAAAGATTATCTCAGCTATTT
TAATGTCTACAGTGATACTTTCTGCTGCAGCCCCGTTGTCAGGTGTTTA
CGCTGACACAAACTCAGATATTGCTAAACAAGATGCGGATTACAAGGAT
GACGACGATAAGGCCTGCCACTCGAGTTGGCAAGATGCACCAATTCAAG
GCACGAGTCAGATGGGCGCACATGGTCAGTTACAGACTTTTCCACGCAA
TGGGTATGACTGGGATAATCAGACACCGCTTGAAGGCGCTGTTTATACG
TTAGTTGATCCATTTGGCCGCCCAATCGTGCCAGGCACCAAGAATGCTT
ATCGCAACTTAGTGTATTATTGTGAGTATCCCGGAGAGCGGCTATATGA
AAACGTCCGTTTTGATGTTAACGGCAATTCACTTGACGAGTACTCCTCT
GATGTCACTACTTTGGTACACCACCACCACCACCACTAA-3′.

The reaction system of overlapping PCR included: 25 μL of 2×pfu-PCR Master Mix, 2.0 μL each of 10 μM/L forward and reverse primers, 1.0 μL of PpepN template, 1.0 μL of Usp45 template, 1.0 μL of p72 template, and supplementing with deionized water to 50 μL.

In the reaction system of overlapping PCR, a forward primer was the forward primer PpepN-F (SEQ ID NO: 6) for amplifying the PpepN promoter fragment, and a reverse primer was the reverse primer P72-R (SEQ ID NO: 11) for amplifying the P72 protein.

PCR reaction procedures included: initial denaturation at 98° C. for 5 min; denaturation at 98° C. for 30 s, annealing at 56° C. for 30 s, and extension at 72° C. for 30 s for a total of 35 cycles; extension at 72° C. for 5 min, and storage at 4° C. after the end of the PCR reaction. The detection results of the PpepN-Usp45-P72 long fragment were shown in FIG. 1C.

### Example 2

Acquisition of the Recombinant Expression Vector pNpepN-ASFV-P72

The cloning vector pNZ8149 and PpepN-Usp45-P72 in Example 1 were double-digested by Nco I and Xbal I, respectively; the double-digested pNZ8149 and PpepN-Usp45-P72 were ligated using T4 ligase, and a ligation product was electrotransformed into a Lactococcus lactis NZ3900 competent cell, and the plasmid in the electrotransformed cell was extracted and sent to Sangon Biotech (Shanghai) Co., Ltd. for sequencing for verification.

The recombinant plasmid was sequenced and aligned with the inserted PpepN-Usp45-P72 gene fragment, and the sequencing result was consistent with expectations. This indicated that the synthesized PpepN-Usp45-P72 gene fragment was successfully inserted into the Lactococcus vector pNZ8149, and the recombinant plasmid was successfully constructed. The positive plasmid was named pNpepN-ASFV-P72, and the plasmid map was shown in FIG. 2.

### Example 3

### Preparation and Detection of ASFV P72 Gene

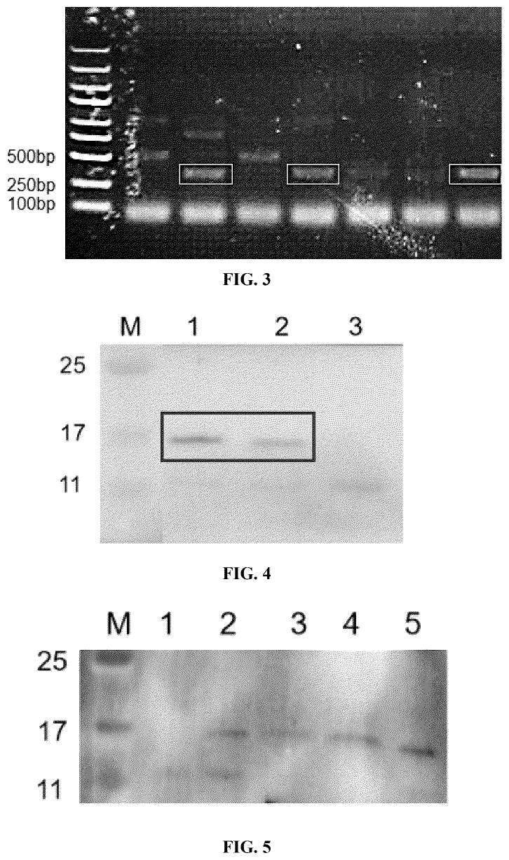

Electrotransformation of the target gene in Lactococcus lactis NZ3900: the electrotransformed Lactococcus lactis NZ3900 was coated on an Elliker solid medium and cultured in a 30° C. incubator for 48 h. The colonies on the medium were selected for PCR identification, where PCR primers were the primers for amplification of P72 gene, and their sequences were set forth in SEQ ID NO: 10 and SEQ ID NO: 11. The identification results were shown in FIG. 3, and positive strain was shown in the box in FIG. 3.

The positive colonies were divided into two groups and inoculated into GM17 liquid medium (a glucose-containing M17 medium), respectively, where one group was added with 10 ng/mL Nisin, recorded as a pNpepN-ASFV-P72 induced group, the other group was not added with Nisin, recorded as a pNpepN-ASFV-P72 non-induced group, and the two groups were cultured at 30° C. for 48 h without shaking. The recombinant strain was verified by Western Blot for the expression of P72 protein.

The culture solution of the cultured bacteria was collected and centrifuged at 8,000 r/min for 10 min to obtain a bacterial sludge, which was then ultrasonically disrupted by an ultrasonic disruptor (Ningbo Xinzhi Biotechnology Co., Ltd., SCIENTZ-1000J, rated power: 1,000 W) at 30% power and a frequency of on 6 s/off 5 s for 10 min in total. After the ultrasonic treatment, a supernatant was collected by centrifugation, and into which an appropriate amount of loading buffer was added for SDS-PAGE electrophoresis; after the electrophoresis, the separated product was transferred onto a PVDF membrane at 80 V for 80 min, followed by blocking with 0.2% gelatin for 2 h, then washed 3 times, incubated with a mouse-derived His tag antibody from Abcam (1:4000 dilution) at 4° C. overnight, washed 3 times the next day, then incubated with an HRP-labeled goat anti-mouse IgG from Solarbio (1:5000 dilution) for 1 h, washed 3 times, and placed in a chemiluminescence imaging system for color development.

The expression of P72 protein by the recombinant strain was verified by Western Blot, and the verification results were shown in FIG. 4. In FIG. 4, lane 1 represents the induced group by Nisin; lane 2 represents the non-induced group; and lane 3 represents the negative control. The Lactococcus recombinant strain has a single band of around 15 kDa. FIG. 4 shows that the band of the induced group by Nisin in lane 1 is thicker than that of the non-induced group in lane 2, while there is no band in the NZ3900 competent cell control group (lane 3). This indicates that the recombinant plasmid pNpepN-ASFV-P72 has been transformed into the NZ3900 competent cells and expressed successfully.

The recombinant Lactococcus lactis was subcultured at an inoculation ratio of 1% by volume, and cultured one generation for every 48 h. The recombinant strain was then verified by Western Blot for the expression of P72 protein, and the results were shown in FIG. 5. In FIG. 5, lane 1 is a negative control; lane 2 is a non-induced recombinant strain of P3 generation; lane 3 is a non-induced recombinant strain of P4 generation; lane 4 is a non-induced recombinant strain of P5 generation; lane 5 is a non-induced recombinant strain of P6 generation. According to FIG. 5, the recombinant Lactococcus had strong stability, and the target protein may be detected even in the strain of the P6 generation.

### Example 4

1. Cultivation Method of Recombinant Lactococcus lactis Strain Expression System

The recombinant Lactococcus lactis strain obtained in Example 3 was inoculated into the Lactococcus lactis GM17 liquid medium at an inoculation ratio of 1% by volume, and a fermentation liquid was harvested after culturing at 30° C. for 12 h. Cascade fermentation was conducted according to an inoculation size of 5% of the total volume, and the recombinant strain was inoculated into 500 mL of MRS liquid medium, 10 L of MRS liquid medium, 200 L of MRS liquid medium in sequence; and after discharging from a fermentation tank (500 L), the fermentation liquid was subjected to colony counting, then a viable count of 3×10¹⁰ CFU/mL was obtained for the fermentation liquid. The fermentation liquid was packaged and stored as an oral live bacteria preparation for preventing ASFV infection.

2. Viable Count of the Recombinant Lactococcus lactis Strain

A plate coating method was adopted:

A, Numbering: 9 sets of sterile plates were marked with markers, 3 sets each of 10⁻⁴, 10⁻⁵, and 10⁻⁶ dilutions. Another 6 test tubes each containing 4.5 mL of sterile water were marked as 10⁻¹, 10⁻², 10⁻³, 10⁻⁴, 10⁻⁵, and 10⁻⁶.

B, Diluting: 1 mL of the thoroughly mixed Lactococcus lactis suspension (sample to be tested) was taken up with a 1 mL sterile pipette, and 0.5 mL of which was accurately placed in the test tube marked with 10⁻¹, which was a 10-fold dilution. The excess bacterial solution was returned to the original bacterial solution. The 10⁻¹ test tube was placed on a test tube oscillator to oscillate the bacterial solution thoroughly. Another 1 mL pipette was inserted into the 10⁻¹ test tube to blow the bacterial suspension three times to further allow the bacterial cells disperse and mixed uniformly. 1 mL of the 10⁻¹ bacterial solution was taken up with this pipette, and 0.5 mL of which was accurately placed in the test tube marked with 10⁻², which was a 100-fold dilution, and the rest were analogous.

C, Sampling: 1 mL of each of the 10⁻⁴, 10⁻⁵, and 10⁻⁶ diluted bacterial suspensions was taken out using three 1 mL sterile pipettes and placed into numbered sterile plates, with 0.2 mL placed in each plate.

D, preparing a solid medium in a petri dish: adding ingredients of a GM17 solid medium into sterile water, mixing, which was subjected to autoclaving, and poured into the petri dish as soon as possible. After the culture medium solidified, 100 μL of the bacterial solutions of different dilutions were added thereon, coated evenly with a sterile coating vessel, then the plate was placed inverted in a constant-temperature incubator at 30° C. for culture.

E, Counting: after 48 h of culture, the culture plates were taken out, and an average number of colonies on three plates with the same dilution was calculated according to the following formula: colony forming unit (CFU) per milliliter=average number of colonies in three replicates of a same dilution×dilution multiple×5.

### Example 5

A farm in Hengnan County, Hengyang City, Hunan Province had 2,500 fattening pigs, with a body size ranging from 20-100 kg. The pigs were raised in two pig houses, A (1,200 pigs) and B (1,300 pigs), respectively. The two pig houses were 50 meters apart, a couple fed the pigs in each house (the couple breeders lived and ate together), and the entire farm adopted fully automatic feeding towers.

On Aug. 24, 2023, it was discovered that a pen of pigs (25 pigs) in House B had fever and decreased feed intake. On August 25, samples from the environment, abnormal pigs, and fan ends in 5 pens (pen Nos. 4/5/6/7/8) having sick pigs were taken and 3 ASFV positive pigs were confirmed. On the same day, all pigs in the two pig houses A and B were fed the recombinant Lactococcus lactis strain prepared in Example 4 through the drinking water system at an amount of 5 mL/pig/day. At the same time, disinfection and precise removal measures were taken, and abnormal pigs were tested for pathogens every day, and positive pigs were immediately removed. On August 26, abnormal pigs in pens 6/7/8 of House B were tested for ASFV pathogens, and 6 positive pigs were found to be positive and immediately removed. By August 28 (the fourth day of feeding the recombinant strain), a total of 28 positive diseased pigs in House B were removed, and no positive pigs were detected in the entire farm thereafter.

During this epidemic, all pigs in the farm were fed recombinant Lactococcus lactis strain at 5 mL/pig/day through drinking water for 21 consecutive days. After the whole herd was stable for 21 d, the recombinant Lactococcus lactis strain was fed in drinking water at 2.5 mL/pig, once every 3 d, for 60 consecutive days. During the whole experimental period, except for the 28 positive pigs that were removed in House B at the early stage of the disease, the rest of the pigs were healthy and without abnormal symptoms. The test results were shown in Table 1.

TABLE 1
Comparison of experimental effects of
recombinant Lactococcus lactis

| Grouping | House A | House B |
|---|---|---|
| Pig herd | Healthy and disease- | 1 pen of pigs showed |
| conditions | free | abnormal symptoms, |
| before | | 3 ASFV positive pigs |
| experiment | | were found |
| Measure | Feed recombinant | Feed recombinant |
| | Lactococcus lactis | Lactococcus lactis |
| | through drinking water | through drinking water for |
| | for a whole process | a whole process combined |
| | | with disinfection and precise |
| | | removal measurements |
| Number of ASFV | 0 | 28 |
| positive pigs | | |
| detected | | |

During this epidemic, the spread of ASF was quickly controlled by feeding recombinant Lactococcus lactis through drinking water in the whole farm, such that there was no epidemic in House A from beginning to end, thereby effectively protecting the health of the pigs in the herd and greatly reducing the economic losses of the pig farm. This indicated that the recombinant Lactococcus lactis strain preparation had a desirable effect.

Although the present disclosure has been described by the above examples in detail, it is only a part of, not all of, the examples of the present disclosure. Other examples may also be obtained by persons based on the examples without creative efforts, and all of these examples shall fall within the protection scope of the present disclosure.

What is claimed is:

1. A dual-promoter recombinant vector expressing an immunogenic protein of an African swine fever virus (ASFV), comprising an original vector and a recombinant gene; wherein the original vector comprises a pNZ8149 vector; the recombinant gene comprises a PpepN promoter, a coding sequence of a Usp45 secretion signal peptide, and a coding sequence of the immunogenic protein of the ASFV, wherein the PpepN promoter, the coding sequence of the Usp45 secretion signal peptide, and the coding sequence of the immunogenic protein of the ASFV are ligated in sequence; and the PpepN promoter comprises the nucleotide sequence set forth in SEQ ID NO: 1 or a complementary sequence of the nucleotide sequence set forth in SEQ ID NO: 1; and
the recombinant gene is located downstream of a promoter in the original vector.

2. The dual-promoter recombinant vector according to claim 1, wherein the Usp45 secretion signal peptide has the amino acid sequence set forth in SEQ ID NO: 2; and
the immunogenic protein of the ASFV comprises an ASFV p72 protein.

3. The dual-promoter recombinant vector according to claim 2, wherein the ASFV p72 protein has the amino acid sequence set forth in SEQ ID NO: 3.

4. The dual-promoter recombinant vector according to claim 1, wherein the recombinant gene is located between restriction sites of Nco I and Xbal I of the original vector.

5. A recombinant bacterium, comprising the dual-promoter recombinant vector according to claim 1; wherein the recombinant bacterium is constructed based on one or more bacteria from a normal intestinal flora.

6. The recombinant bacterium according to claim 5, wherein the normal intestinal flora is selected from the group consisting of Bifidobacterium, Lactococcus, Lactobacillus, and Streptococcus.

7. The recombinant bacterium according to claim 6, wherein the Lactococcus comprises Lactococcus lactis NZ3900.

8. A microbial preparation for preventing ASFV infection, comprising the recombinant bacterium according to claim 5 as an active ingredient.

9. The microbial preparation according to claim 8, wherein the recombinant bacterium has a viable count of 2×10¹⁰ CFU/mL to 3×10¹⁰ CFU/mL.

10. A method for preventing ASFV infection, comprising
administrating the dual-promoter recombinant vector according to claim 1 to a subject in a risk of infection by the ASFV.

11. The method according to claim 10, wherein the Usp45 secretion signal peptide has the amino acid sequence set forth in SEQ ID NO: 2; and
the immunogenic protein of the ASFV comprises an ASFV p72 protein.

12. The method according to claim 11, wherein the ASFV p72 protein has the amino acid sequence set forth in SEQ ID NO: 3.

13. The method according to claim 10, wherein the recombinant gene is located between restriction sites of Nco I and Xbal I of the original vector.

14. A method for preventing ASFV infection, comprising
administrating the recombinant bacterium according to claim 5 to a subject in a risk of infection by the ASFV.

15. The method according to claim 14, wherein the normal intestinal flora is selected from the group consisting of Bifidobacterium, Lactococcus, Lactobacillus, and Streptococcus.

16. The method according to claim 15, wherein the Lactococcus comprises Lactococcus lactis NZ3900.

17. A method for preventing ASFV infection, comprising
administrating the microbial preparation according to claim 8 to a subject in a risk of infection by the ASFV.

18. The method according to claim 17, wherein the recombinant bacterium has a viable count of 2×10¹⁰ CFU/mL to 3×10¹⁰ CFU/mL.
